(19) United States
(12) Patent Application Publication
(10) Pub. No.: US 2020/0022714 A1
(43) Pub. Date: Jan. 23, 2020

(54) ULTRASOUND DEVICES INCORPORATING PHASE CHANGE MATERIALS AND SYSTEMS AND METHODS USING THE DEVICES

(71) Applicant: SonoMotions Inc., South San Francisco, CA (US)

(72) Inventor: Paul Douglas Corl, Palo Alto, CA (US)

(21) Appl. No.: 16/328,660

(22) PCT Filed: Oct. 13, 2017
(86) PCT No.: PCT/US2017/056635

§ 371 Date: Feb. 26, 2019

Related U.S. Application Data
(60) Provisional application No. 62/409,803, filed on Oct. 18, 2016.

(51) Int. Cl. A61B 17/22

(52) U.S. Cl. CPC A61B 17/2202; A61B 2017/22024; A61B 2017/22027

(57) ABSTRACT

An ultrasound therapy probe includes a housing; a therapy transducer disposed in the housing to produce acoustic waves for therapy; a lens coupled to the housing to focus the acoustic waves from the therapy transducer for delivery to a patient; and a phase change material disposed within the housing and in thermal communication with the therapy transducer. The phase change material has a phase transition temperature or temperature range within a range of 25° C. to 40° C. at which the phase change material changes from a first phase to a second phase, where the first and second phases are not gaseous phases. The phase change material forms a thermal reservoir for managing thermal energy arising from the ultrasound therapy probe.

### CROSS-REFERENCE TO RELATED APPLICATIONS

This application claims the benefit under 35 U.S.C. § 119(e) of U.S. Provisional Patent Application Ser. No. 62/409,803 filed Oct. 18, 2016, which is incorporated herein by reference.

### FIELD

The present invention is directed to the area of ultrasound devices. The present invention is also directed to ultrasound devices, systems, and methods for the treatment of urinary stone disease and other disorders.

### BACKGROUND

Urinary Stone Disease (USD) affects adults and children. According to one estimate, one in eleven Americans now has USD and that number is increasing. In many instances, the methods of treatment include the expulsion of kidney stones from the patient. Residual fragments are widely considered to be the overwhelming clinical and research priority in USD, because current treatment options, such as shock wave lithotripsy (SWL) or ureteroscopic lithotripsy (URS), leave behind small residual stone fragments. Studies have shown that while most residual stone fragments will pass, others may grow and in approximately 20% to 40% of patients, lead to symptomatic events such as pain, emergency room visits, or additional procedures. One current treatment is the serial use of focused ultrasound to manipulate one stone at a time. However, for a large number of small stones this serial method can be unfeasible or too expensive. There is a need for additional devices, techniques, and methods for treating USD and other related disorders or diseases.

### BRIEF SUMMARY

One embodiment is an ultrasound therapy probe that includes a housing; a therapy transducer disposed in the housing and configured and arranged to produce acoustic waves for therapy; a lens coupled to the housing and configured and arranged to focus the acoustic waves from the therapy transducer for delivery to a target region within a patient; and a phase change material disposed within the housing and in thermal communication with the therapy transducer. The phase change material has a phase transition temperature or temperature range within a range of 25° C. to 40° C. at which the phase change material changes from a first phase to a second phase, where the first and second phases are not gaseous phases. The phase change material forms a thermal reservoir for managing thermal energy arising from the ultrasound therapy probe.

Another embodiment is an ultrasound therapy probe that includes a housing; a therapy transducer disposed in the housing and configured and arranged to produce acoustic waves for therapy; a lens coupled to the housing and configured and arranged to focus the acoustic waves from the therapy transducer for delivery to a target region within a patient; and a phase change material disposed within the housing and in thermal communication with the therapy transducer. The phase change material has a phase transition temperature or temperature range within a range of 25° C. to 40° C. at which the phase change material changes from a first solid or liquid phase to a second solid or liquid phase. The phase change material forms a thermal reservoir for managing thermal energy arising from the ultrasound therapy probe.

In at least some embodiments, the ultrasound therapy probe further includes an imaging probe disposed within the housing. In at least some embodiments, the first phase is a solid phase and the second phase is a liquid phase. In at least some embodiments, the first phase is a first solid phase and the second phase is a second solid phase. In at least some embodiments, the phase transition temperature or temperature range is within a range of 30° C. to 37° C. or in a range from 33° C. to 36° C.

In at least some embodiments, the ultrasound therapy probe further comprises a chamber disposed within the housing and acoustically isolated from the transducer, where the phase change material is disposed within the chamber with a thermally conductive pathway connecting the phase change material to the therapy transducer. In at least some embodiments, the phase change material is in direct contact with the therapy transducer. In at least some embodiments, the housing includes a transducer chamber within which the therapy transducer and the phase change material are disposed.

In at least some embodiments, the phase change material includes a paraffin, a fatty acid, a salt hydrate, a eutectic mixture, or any combination thereof. In at least some embodiments, the phase change material includes a micro-encapsulated phase change material. In at least some embodiments, the micro-encapsulated phase change material forms at least a portion of the housing. In at least some embodiments, the phase change material has a volume of at least 5 cm³.

Yet another embodiment is a method of using any of the ultrasound therapy probes described above to provide therapy to a single patient at a single therapy session. The method includes operating the therapy transducer for a first period of time causing the phase transition material to at least partially transition from the first phase to the second phase; resting the therapy transducer for a second period of time allowing heat to dissipate from the phase transition material causing a transition of at least a portion of the phase transition material from the second phase to the first phase; and operating the therapy transducer for a third period of time causing the phase transition material to at least partially transition from the first phase to the second phase.

In at least some embodiments, resting the therapy transducer for a second period of time includes placing at least a portion of the ultrasound therapy probe into a cooling arrangement to facilitate dissipation of the heat. In at least some embodiments, the cooling arrangement is an ice bath or refrigerator.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a top perspective view of one embodiment of an ultrasound therapy probe;

FIG. 2 is an exploded view of the ultrasound therapy probe of FIG. 1;

FIG. 3 is a cross-sectional view of the ultrasound therapy probe of FIG. 1;

FIG. 4A is a cross-sectional view of a portion of one embodiment of an ultrasound therapy probe containing a phase change material, according to the invention;

FIG. 4B is a cross-sectional view of a portion of another embodiment of an ultrasound therapy probe containing a phase change material, according to the invention; and

FIG. 4C is a cross-sectional view of a portion of a third embodiment of an ultrasound therapy probe containing a phase change material, according to the invention.

### DETAILED DESCRIPTION

The present invention is directed to the area of ultrasound devices. The present invention is also directed to ultrasound devices, systems, and methods for the treatment of urinary stone disease and other disorders.

Any suitable ultrasound device (often referred to as an ultrasound probe) for treating urinary stone disease or other disorders can be modified as described herein to incorporate phase change materials. Examples of suitable ultrasound devices or probes are disclosed in U.S. Pat. No. 9,204,859 and PCT Patent Application Publication No. WO 2016/061587, both of which are incorporated herein by reference. These references also disclose methods and techniques for providing ultrasound therapy to, for example, move or break up kidney stones or fragments thereof. In addition to a therapy transducer, these devices may also incorporate an imaging probe that can use, for example, ultrasound imaging to monitor and display the locations of the stones or stone fragments and to monitor the progress of the therapy.

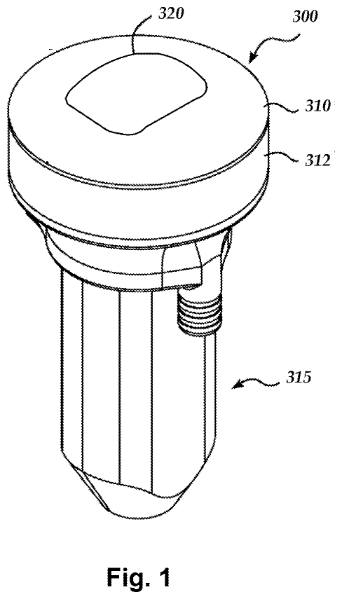

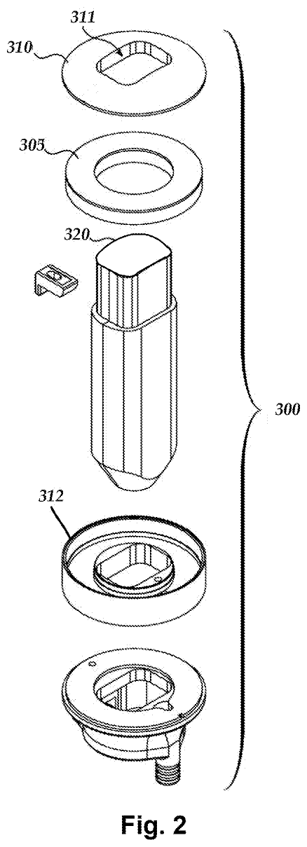

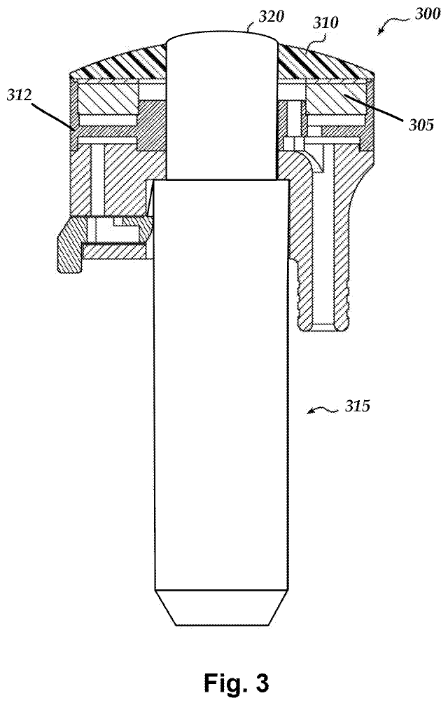

FIGS. 1-3 illustrate one embodiment of an ultrasound device or probe from PCT Patent Application Publication No. WO 2016/061587 that can be modified as described herein to incorporate phase change materials. The ultrasound therapy probe 300 includes a therapy transducer 305, a lens 310, a transducer housing 312, an imaging probe 320, which fits through the lens 310 and therapy transducer 305 via an aperture 311, also providing an elongate handle 315 for the therapy/imaging probe combination.

An ultrasound therapy probe uses transducer(s) that produce high-intensity focused ultrasound waves to generate acoustic radiation pressure (for moving kidney stones) or other mechanical effects (for breaking kidney stones). A key limiting factor for the acoustic performance is probe heating due to the power dissipation in the transducer and mechanical losses in the lens and housing structures.

As an example, in at least some embodiments, the pulse-average electrical power delivered to the transducer may range from 100 W to 4 kW, depending on the application (e.g., moving or breaking stones). The time-average power dissipation may be moderated by utilizing a low duty-cycle electrical excitation.

One approach to managing probe heating while maintaining acoustic output is to design an efficient ultrasound transducer so that most of the electrical power delivered to the probe is converted into acoustic power that propagates into the tissue. However, practically speaking, it is difficult to achieve transducer electrical-to-mechanical conversion efficiency greater than about 85% in the frequency range from 100 kHz to 1 MHz which is often used for these techniques. Moreover, overall probe efficiency is typically in the range from 50% to 75%, after including the losses in the lens and the housing structures.

Another approach to moderate probe heating is to use a low duty cycle electrical excitation to reduce the time-average electrical power, while maintaining the high pulse-average power for treatment. However, if the duty cycle is reduced, procedure time may be increased.

Conventionally, heat generated by some devices is dissipated, at least in part, using a heat sink, such as a finned structure, that uses increased surface area for convective cooling to the surrounding air. However, within the constraints of a practical probe geometry for the stone moving/breaking application, there is limited potential for this approach. Increasing the size of the probe may make it difficult to grasp and position, and intricate fin structures preferred for efficient cooling are subject to fouling by the ultrasound coupling gel.

Adding a fan or active cooling (Peltier device) has limited potential for this application as well. A fan would be subject to fouling by the ultrasound gel which typically ends up covering the entire probe by the end of a procedure. An active cooling device only moves the “hot” spot from one place to another while adding more heat due to its inefficiency. A hot spot created by active cooling would need to be protected from contact with the patient or physician to prevent the possibility of a burn.

Another approach for cooling a probe would be to circulate a cooling fluid (gas or liquid) through the device to a remote heat sink that would be cooled efficiently. This arrangement can be effective, but it adds complexity to the cable and connector assembly associated with the probe, which must now carry electrical signals as well as fluid flow conduits.

In contrast to these approaches, the present devices, systems, and methods store thermal energy in a reservoir within the probe for a period of time, such as the duration of a clinical procedure (or one part of a longer clinical procedure). Such an arrangement can be used by itself or in combination with any of the other approaches discussed above.

The reservoir contains a phase change material (PCM) which is typically a solid material when the procedure begins and changes phase (for example, from solid to liquid or from one solid phase to another solid phase) as the material is heated during the procedure. Such phase change materials (PCMs) can store a large amount of thermal energy with only a small or no change in temperature. At least a portion of the thermal energy is used to convert the PCM from one phase to another.

Materials not undergoing a phase change can provide a thermal mass or reservoir for storing thermal energy, but the temperature of the thermal mass will rise at a rate approximately proportional to the thermal energy. One of the best materials for storing thermal energy is water, with a volumetric heat capacity of 4.19 J/° Kcm³. Most solid materials have lower volumetric heat capacity, with the added disadvantage of being heavier. For a temperature rise on the order of 25° C. above ambient, a water reservoir would store ˜100 J/cm³.

In contrast, paraffin is one example of a suitable phase change material for an ultrasonic probe. One exemplary material, a 20-carbon paraffin (C₂₀H₄₂) has a melting point of about 36.7° C., and a latent heat of fusion of approximately 200 J/cm³ to change the phase of the paraffin with little or no change in temperature.

Another example of a suitable phase change material for an ultrasonic probe is the decahydrate of sodium sulfate (Na₂SO₄.10H₂O). This material has a phase transition temperature of about 32.4° C., and a latent heat of fusion of approximately 370 J/cm³ to change the phase of the salt-hydrate with little or no change in temperature.

Another example of a suitable phase change material for an ultrasonic probe is the commercial product savE® OM37 from Pluss Advanced Technologies Pvt. Ltd. This material has a melting point of about 37° C., and a latent heat of fusion of approximately 180 J/cm³ to change the phase of the material with little or no change in temperature.

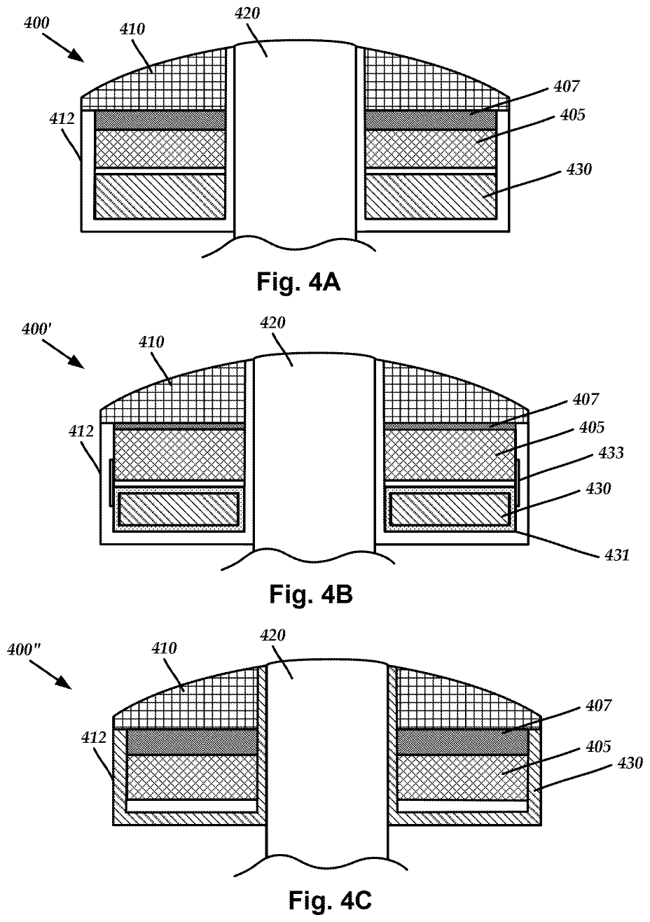

FIG. 4A illustrates, in cross-section, a portion of an ultrasound therapy probe 400 with a therapy transducer 405, an optional matching layer 407, a lens 410, a transducer housing 412, and an imaging probe 420. In addition, the ultrasound therapy probe 400 includes a phase change material 430 that is in thermal communication with the probe transducer 405. In the illustrated embodiment, there is separation between the phase change material 430 and the transducer 405. The separation may be an air gap or there may be a layer or other barrier between the transducer and the phase change material facilitating thermal communication between the transducer and the phase change material. Such a layer or barrier may protect or separate the transducer 405 from the phase change material 430 or may be, for example, a backing material, that prevents, ameliorates, or modifies acoustic effects that would otherwise be associated with the phase change material. In yet other embodiments, the phase change material 430 may be in contact with the transducer.

In at least some embodiments, the transducer housing 412 forms a single transducer chamber in which the therapy transducer 405 and the phase change material 430 are disposed. In some embodiments the phase change material 430 fills the space within the transducer housing 412 not occupied by the transducer 405, but in other embodiments, such as the illustrated embodiment, there may be additional space (for example, at least 5%, 10%, 20%, 25%, 30%, 40%, or 50%, or more of the space defined by the housing and other components) within the transducer housing to, for example, allow for density changes of the phase change material 430 or to separate the phase change material from the transducer 405. Typically, the phase transition of the phase change material 430 during operation of the ultrasound probe is from a solid or liquid phase to another solid or liquid phase. Such transitions typically have relatively small changes in density. In contrast, phase transitions from solid or liquid phase to a gaseous phase are generally not suitable because of the large difference in density between the solid/liquid and gas.

In at least some embodiments, the phase change material has a phase change temperature in the range of 25° C. to 40° C. or the range of 30° C. to 37° C. or the range of 33° C. to 36° C. In at least some embodiments, a phase change temperature of less than 25° C. (for example, in the range of 15° C. to 25° C. or in the range of 10° C. to 25° C.) can be used if the probe is cooled prior to use, although such embodiments may feel too cold to a patient if applied to the skin unless the phase change material is at least partially thermally insulated from the housing of the device.

In at least some embodiments, the volume of phase change material is at least 5 cm³. For example, the volume can be in a range from 5 to 50 cm³ or in a range from 10 to 40 cm³ or in a range from 15 to 30 cm³. It will be recognized, however, that some applications can use smaller or larger volumes of phase change material. The volume of phase change material may depend on factors such as the size of the device, the typical duration of treatment, the typical energy supplied to or by the transducer during treatment, and the like.

It will be recognized that some suitable phase change materials have phase changes that do not exhibit sharp temperature points (for example, some phase changes from ordered to amorphous phases), but may occur over a range of temperatures (for example, over a range of 1, 2, 3, 4, 5, or more degrees Celsius.) In some embodiments, the phase change material undergoes a change from a solid phase to a liquid phase such as, for example, paraffin. In some embodiments, the phase change may be from a more ordered phase to a less ordered phase, such as, for example, from a crystalline to an amorphous phase. Polymeric materials are examples of materials that can have multiple solid phases.

Examples of suitable phase change material include, but are not limited to, organic materials such as paraffins (different carbon chain lengths provide different melting temperatures) or fatty acids and esters (such as palmitic acid or capric acid), inorganic materials including salt hydrates (such as Na₂SO₄.10H₂O), eutectic mixtures of various compounds, and a wide range of proprietary formulations from manufacturers including Entropy Solutions LLC, Rubitherm Technologies GmbH, Pluss Advanced Technologies Pvt. Ltd., and Phase Change Material Products Limited. Some phase change materials are available in a micro-encapsulated format (for example microscopic beads of phase change material encapsulated in a polymer shell). Examples of these micro-encapsulated phase change materials are available from companies including Microtek Laboratories, Inc., or BASF SE. In at least some embodiments, the choice of phase change material may take into consideration one or more properties such as, for example, the phase transition temperature (or temperature range), the heat of fusion (or of other phase change), the safety profile (for example, flammability or toxicity), or other factors affecting the suitability for incorporation into the particular therapy probe design, or any combination of these factors.

Preferably, the phase transition is reversible. For example, after paraffin melts in a liquid state during a procedure, the paraffin can be returned to the solid state by allowing the probe to cool or even placing the probe in or near ice or in a refrigerator or other cooling device. Reversibility of the phase transition of the phase change material facilitates reuse of the probe. In some embodiments of operation, the probe 400 can be used for a first period of time and then allowed to cool in air or placed in an ice bath, refrigerator, or other cooling apparatus for a second period of time, and then the probe 400 can be used again to continue the procedure. This process may be repeated any number of times during the entire duration of the procedure. For example, the first period of time may be in the range of 1 to 10 minutes or more, followed by 1 to 10 minutes or more of cooling, with the process being repeated as needed. Air cooling may take longer, for example, 30 minutes to 60 minutes or longer. These periods of time are simply examples. In some embodiments, the periods of time for a procedure and for cooling may be longer or shorter. As indicated above, other thermal management techniques, such as, for example, duty cycle limitations, may be utilized in conjunction with the phase change material to enhance the period time for operation of the ultrasound probe.

Preferably, the phase change material 430 is not chemically reactive with surrounding materials, such as the transducer 405 or transducer housing 412, at the temperature and pressures achieved during operation. Preferably, the phase change material 430 is non-conductive, particularly if the phase change material may be in contact with (or may inadvertently make contact with) the transducer 405. Preferably, the phase change material 430 does not diffuse into the piezoelectric material of the transducer 405 or any other material associated with the transducer 405 and in contact with the phase change material 430 in order to avoid or reduce changes in the acoustic properties of the probe over time.

Preferably, the transducer housing 412 and other portions of the probe 400 in contact with the phase change material 430 are arranged to prevent or reduce leaking of the phase change material 430 during or after operation. In at least some embodiments, the phase change material 430 may transition to a liquid or partially liquid state and the probe 400 is preferably designed to prevent or reduce leakage of the liquid phase change material.

Any suitable transducer can be used for the therapy transducer 405 including, but not limited to, lead zirconate titanate (PZT) transducers or transducers formed using other piezoelectric materials. Any suitable imaging probe 420 can be used including commercially available imaging probes. In at least some embodiments, the therapy transducer 405 has a frequency in a range of 100 kHz to 1 MHz or higher. In at least some embodiments, the ultrasound therapy probe 400 is configured to deliver an average acoustic power of 5 W to 200 W for 1 second to 10 min or an average acoustic power of 10 W to 200 W for 1 second to 10 min or an average acoustic power of 15 W to 60 W for 1 second to 10 min. In at least some embodiments, an outer dimension of the therapy transducer 405 is in a range of 3 to 15 cm or in a range of 4 to 12 cm or in a range of 5 to 10 cm. In at least some embodiments, an inner dimension of the therapy transducer 405 is in a range of 1 to 10 cm or in a range of 3 to 5 cm. The therapy transducer 405 is coupled to a pulse generator or amplifier, which provides the electrical excitation to the transducer to produce the desired acoustic output.

The lens 410 facilitates focusing of acoustic waves generated by the therapy transducer into a target treatment zone within the patient (e.g., within the kidney or the ureter for the treatment of kidney stones). The lens may have a flat, convex, or concave exterior surface. The lens 410 can be formed of any suitable material including, but not limited to, plastic, oil, ceramic, alcohol, water based fluid, gel, metal (e.g. aluminum), graphite, or any combination thereof. Examples of suitable plastics include, but are not limited to, polysiloxanes and polyurethanes.

Any suitable matching layer 407 can be used or the transducer 405 can be positioned adjacent to the lens 410. In at least some embodiments, a matching layer is disposed between the therapy transducer 405 and the lens 410 to facilitate transition of acoustic impedance between the transducer and the lens. Examples of suitable materials for a matching include, but are not limited to, graphite or composites, such as an epoxy loaded with tungsten, alumina, or other particulate material.

The transducer housing 412 can be made out of any suitable material including plastics or metals or any combination thereof.

FIG. 4B illustrates, in cross-section, a portion of another ultrasound therapy probe 400′ with a therapy transducer 405, an optional matching layer 407, a lens 410, a transducer housing 412, and an imaging probe 420. In addition, the ultrasound therapy probe 400′ includes a chamber 431 within which a phase change material 430 is disposed. The probe 400′ also optionally contains a thermal conduction bridge 433 to facilitate thermal communication between the chamber 431/phase change material 430 and the probe transducer 405.

The chamber 431 forms a housing around the phase change material 430 and can be made of any thermally conductive material including, but not limited to metals or alloys or thermally conductive plastics. For example, the chamber 431 can be made of copper. Plastics may be used for the chamber although heat conduction may be less for such materials. Thus, the chamber 431 retains the phase change material 430 and facilitates avoiding contact between the phase change material 430 and the transducer 405. For example, if the phase change material 430 liquefies or softens, it may flow and could contact the transducer 405 absent the chamber 431. In some embodiments, the chamber 431 surrounds the phase change material. In other embodiments, the chamber 431 may simply provide a solid barrier between the phase change material 430 and the transducer 405. In some embodiments, a metal mesh or metal wool (such as copper mesh or copper wool) or other conductive arrangement may be disposed within the phase change material 430 (for example, in the embodiments of either 4A or 4B) to facilitate distribution of heat through the phase change material.

The optional thermal conduction bridge 433 facilitates heat transfer from the transducer 405 to the chamber 431 and phase change material 430. In at least some embodiments, at least the portion of the thermal conduction bridge 433 in contact with or near the transducer 405 is made of a non-electrically-conductive material to avoid shorting the front and back electrodes of the transducer or to prevent electrical conduction between the back electrode of the transducer with the chamber 431 or phase change material 430. Examples can include thermally conductive glue or adhesive. In some embodiments, a portion of the thermal conduction bridge 433 in contact with or near the chamber 431 can be made of electrically conductive materials, such as metals or alloys, although non-electrically-conductive materials may also be used. In at least some embodiments, the chamber 431 or phase change material 430 is electrically grounded. The optional thermal conduction bridge may also be used in the embodiment illustrated in FIG. 4A to facilitate conduction between the transducer 405 and the phase change material 430.

FIG. 4C illustrates, in cross-section, a portion of yet another embodiment of an ultrasound therapy probe 400″ with a therapy transducer 405, an optional matching layer, a lens 410, a transducer housing 412, and an imaging probe 420. In addition, the ultrasound therapy probe 400″ includes a phase change material 430 that forms at least a portion of the housing 412 and is in thermal communication with the probe transducer 405.

For example, some phase change materials are available in a micro-encapsulated format (for example microscopic beads of phase change material encapsulated in a polymer shell) that can be incorporated into the polymer material that forms the body of a therapy probe housing. Examples of these micro-encapsulated phase change materials are available from companies including Microtek Laboratories, Inc., or BASF SE. A composite material of a polymer matrix filled with micro-encapsulated phase change material beads can then be part of the transducer housing 412. Other phase change materials may also be used as part of the transducer housing 412 so long as the integrity of the housing is not compromised by the phase change of the phase change material.

In the illustrated embodiment, the entire transducer housing 412 incorporates the phase change material. In other embodiments, only one or more portions of the transducer housing 412 incorporate the phase change material. It will also be recognized that phase change material can also be used in other portions of the therapy probe 400, 400′, 400″, in the imaging probe 420, or any combination thereof

It will be recognized that the different arrangements of the phase change material 430 illustrated in FIGS. 4A-4C can be used in any combination. For example, one embodiment can include a phase change material 430 in a chamber, as in FIG. 4B, as well as a phase change material within the transducer housing 412, as in FIG. 4C. These phase change materials may be the same or different materials.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An ultrasound therapy probe, comprising:
a housing;
a therapy transducer disposed in the housing and configured and arranged to produce acoustic waves for therapy;
a lens coupled to the housing and configured and arranged to focus the acoustic waves from the therapy transducer for delivery to a target region within a patient; and
a phase change material disposed within the housing and in thermal communication with the therapy transducer, the phase change material having a phase transition temperature or temperature range within a range of 25° C. to 40° C. at which the phase change material changes from a first phase to a second phase, wherein the first and second phases are not gaseous phases, wherein the phase change material forms a thermal reservoir for managing thermal energy arising from the ultrasound therapy probe.

2. The ultrasound therapy probe of claim 1, further comprising an imaging probe disposed within the housing.

3. The ultrasound therapy probe of claim 1, wherein the first phase is a solid phase and the second phase is a liquid phase.

4. The ultrasound therapy probe of claim 1, wherein the first phase is a first solid phase and the second phase is a second solid phase.

5. The ultrasound therapy probe of claim 1, wherein the phase transition temperature or temperature range is within a range of 30° C. to 37° C.

6. The ultrasound therapy probe of claim 1, further comprising a chamber disposed within the housing and acoustically isolated from the transducer, wherein the phase change material is disposed within the chamber with a thermally conductive pathway connecting the phase change material to the therapy transducer.

7. The ultrasound therapy probe of claim 1, wherein the housing comprises a transducer chamber within which the therapy transducer and the phase change material are disposed.

8. The ultrasound therapy probe of claim 1, wherein the phase change material comprises a paraffin, a fatty acid, a salt hydrate, a eutectic mixture, or any combination thereof.

9. The ultrasound therapy probe of claim 1, wherein the phase change material comprises a micro-encapsulated phase change material.

10. The ultrasound therapy probe of claim 9, wherein the micro-encapsulated phase change material forms at least a portion of the housing.

11. An ultrasound therapy probe, comprising:
a housing;
a therapy transducer disposed in the housing and configured and arranged to produce acoustic waves for therapy;
a lens coupled to the housing and configured and arranged to focus the acoustic waves from the therapy transducer for delivery to a target region within a patient; and
a phase change material disposed within the housing and in thermal communication with the therapy transducer, the phase change material comprises a phase transition temperature or temperature range within a range of 25° C. to 40° C. at which the phase change material changes from a first solid or liquid phase to a second solid or liquid phase, wherein the phase change material forms a thermal reservoir for managing thermal energy arising from the ultrasound therapy probe.

12. The ultrasound therapy probe of claim 11, further comprising an imaging probe disposed within the housing.

13. The ultrasound therapy probe of claim 11, wherein the first solid or liquid phase is a solid phase and the second solid or liquid phase is a liquid phase.

14. The ultrasound therapy probe of claim 11, further comprising a chamber disposed within the housing and acoustically isolated from the transducer, wherein the phase change material is disposed within the chamber with a thermally conductive pathway connecting the phase change material to the therapy transducer.

15. The ultrasound therapy probe of claim 11, wherein the housing comprises a transducer chamber within which the therapy transducer and the phase change material are disposed.

16. The ultrasound therapy probe of claim 11, wherein the phase change material comprises a micro-encapsulated phase change material that also forms a portion of the housing.

17. The ultrasound therapy probe of claim 11, wherein the phase change material comprises a paraffin, a fatty acid, a salt hydrate, a eutectic mixture, or any combination thereof.

18. A method of using the ultrasound therapy probe of claim 1 to provide therapy to a single patient at a single therapy session, the method comprising:
operating the therapy transducer for a first period of time causing the phase transition material to at least partially transition from the first phase to the second phase;
resting the therapy transducer for a second period of time allowing heat to dissipate from the phase transition material causing a transition of at least a portion of the phase transition material from the second phase to the first phase; and
operating the therapy transducer for a third period of time causing the phase transition material to at least partially transition from the first phase to the second phase.

19. The method of claim 18, wherein resting the therapy transducer for a second period of time comprises placing at least a portion of the ultrasound therapy probe into a cooling arrangement to facilitate dissipation of the heat.

20. The method of claim 19, wherein the cooling arrangement is an ice bath or refrigerator.
